(19) United States
(12) Patent Application Publication
(10) Pub. No.: US 2011/0008413 A1
(43) Pub. Date: Jan. 13, 2011

(54) Compositions and Methods of Topical Drug Delivery for the Treatment of Carpal Tunnel Syndrome

(75) Inventors: Yong Qiu, San Diego, CA (US); James S. Kuo, San Diego, CA (US); Geraldine P. Kuo, San Diego, CA (US); Chin-Chih Chiang, San Diego, CA (US); Paul R. Chen, San Diego, CA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: MSK PHARMA, LLC, San Diego, CA (US)

(21) Appl. No.: 12/831,717

(22) Filed: Jul. 7, 2010

Related U.S. Application Data
(60) Provisional application No. 61/223,975, filed on Jul. 8, 2009.

(51) Int. Cl. A61K 9/70; A61K 31/573; A61K 31/58; A61K 31/57; A61K 31/616; A61P 23/02; A61P 29/00

(52) U.S. Cl. 424/448; 514/171; 424/449; 514/161

(57) ABSTRACT

The present invention generally relates to transdermal drug delivery systems. More particularly, the present invention provides compositions and transdermal drug delivery systems for the treatment and/or relief of symptoms associated with carpal tunnel syndrome or tendonitis.

### RELATED APPLICATION

This application claims the benefit of U.S. Provisional Application No. 61/223,975, filed Jul. 8, 2009, which is incorporated herein by reference.

### FIELD OF THE INVENTION

The present invention generally relates to transdermal drug delivery systems. More particularly, the present invention provides methods and compositions of transdermal drug delivery systems for the relief of symptoms associated with carpal tunnel syndrome or tendonitis.

### BACKGROUND OF THE INVENTION

Carpal tunnel syndrome is the most common mononeuropathy of the upper extremity caused by elevated pressure and subsequent compression of the median nerve at the wrist. The carpal tunnel is a narrow, rigid passageway located in the wrist formed by carpal bones on one side and the transverse carpal ligament on the other side. The median nerve (along with nine flexor tendons) passes through the carpal tunnel into the hand and supplies sensation to the palmar aspect of the thumb, index finger, middle finger and the radial half of the ring finger and movement of the thenar muscles of the thumb. The most common cause of carpal tunnel syndrome is nonspecific flexor tenosynovitis that leads to swelling within the carpal tunnel. As the dimensions of the carpal tunnel are fixed, any increase in tissue size within the carpal tunnel can cause compression of the median nerve. Compression of the median nerve leads to ischemia of the nerve and its dysfunction.

Carpal tunnel syndrome is predominately found in women 30 to 60 years old, but it is also found in men and in all age groups. Some of the conditions associated with carpal tunnel syndrome include pregnancy, premenstrual syndrome (PMS), and menopause; this is probably because of hormone changes that cause fluid retention and swelling of the tissues. Other conditions associated with carpal tunnel syndrome include sprain or fracture of the wrist, rheumatoid arthritis, renal failure, diabetes mellitus, acromegaly, hypothyroidism, multiple myeloma, obesity, recent tuberculosis, fungal infection, and high blood pressure. Injury or trauma to the area, including (but not limited to) repetitive movement of the wrists, can cause swelling of the tissues and carpal tunnel syndrome. This injury may be from sports such as racquetball and handball, or from sewing, typing, driving, assembly-line work, painting, writing, use of tools (especially hand tools or tools that vibrate), repetitive stress or movement, or similar activities.

Carpal tunnel syndrome is characterized by the presence of one or more of the following symptoms: (a) atrophy or weakness in one or both hands; (b) numbness, burning, tingling, paraesthesia or pain in the thumb, index, middle, and the radial half of the ring fingers of one or both hands; (c) the above symptoms may radiate to the wrist, forearm, or shoulder; (d) impaired fine finger movements or clumsiness in one or both hands; (e) weak grip or dropping of objects; and (f) difficulty bringing the thumb across the palm to meet the other fingers (thumb opposition).

Treatment for carpal tunnel syndrome varies depending on the severity of the condition. The current treatment options included reducing or modifying the offending activity; wrist splinting; oral non-steroidal anti-inflammatory drugs (NSAIDs); oral synthetic glucocorticoids; injection of the carpal tunnel with synthetic local anesthetics and/or synthetic glucocorticoids; and surgery.

The increasing prevalence of carpal tunnel syndrome, particularly due to increasing work related repetitive stress injuries, has led to continued interest in identifying new methods for treating this condition. Of interest would be a simple method for relieving one or more of the symptoms associated with carpal tunnel syndrome that could be self-administered and would have minimal systemic effects.

Transdermal drug delivery is a comfortable, convenient, and noninvasive way of administering drugs. The variable rates of absorption and metabolism associated with oral treatment are avoided, as well as eliminating other inherent inconveniences such as gastrointestinal irritation and the like. When the therapeutic compounds are delivered transdermally, the blood concentrations of the drugs can be highly controlled because of the constant flux rate at the steady state. These advantages make transdermal drug delivery a preferred route for treating carpal tunnel syndrome.

Having many advantages, however, one of the key problems with transdermal administration has been the low penetration or permeation rate of many drugs through the skin of the patient. Skin is a structurally complex, relatively thick membrane. Molecules penetrating from the environment into and through the intact skin must first penetrate the stratum corneum, and then penetrate the viable epidermis, the papillary dermis, and the capillary walls into the bloodstream or lymph channels. Transport across the skin membrane is thus a complex phenomenon. It posts great challenges to develop transdermal drug delivery systems.

To develop an effective, noninvasive and convenient topical formulation for carpal tunnel syndrome, the current effective therapeutic methods must be taken as a reference. One such existing method to treat carpal tunnel syndrome is to inject 1 mL of 1% lidocaine and 1 mL of steroid (40 mg triamcinolone) with a 25 gauge needle into the carpal tunnel space. Usually this procedure is done only by a specialist physician, such as a hand surgeon, rheumatologist, or physical medicine and rehabilitation physician. Multiple clinical studies have demonstrated that injection into the carpal tunnel space with steroids is more effective than oral steroids in relieving symptoms associated with carpal tunnel syndrome. Often, lidocaine is co-injected with the steroid to allow immediate pain relief and reduction in paresthesias and pain. Therefore, it is reasoned that a topical formulation consisting of synthetic local anesthetic and/or synthetic glucocorticoids and/or non-steroidal anti-inflammatory agents should optimally relieve one or more of the symptoms associated with carpal tunnel syndrome. Such a topical formulation can mimic the current injection therapy, but do so in a noninvasive manner and thus avoid anxiety, pain, complication and cost associated with injection. However, the well known difficulties to transdermally deliver steroids due to their low penetration or permeation rate post a great challenge to develop such topical formulations. Furthermore, transdermal drug delivery systems with more than one drug are generally more difficult to formulate in view of different interactions with each drug and the carrier, excipients, etc.

In view of the foregoing, it will be appreciated that providing compositions and methods for topical drug delivery to treat carpal tunnel syndrome effectively, noninvasively, conveniently and comfortably would be a significant advancement in the art.

### SUMMARY OF THE INVENTION

It is an object of the present invention to overcome the difficulties and disadvantages of the known art in treating carpal tunnel syndrome described above. Another object of the invention is to provide the compositions and methods for a transdermal delivery system consisting of synthetic local anesthetic and/or synthetic glucocorticoids and/or non-steroidal anti-inflammatory agents, which could optimally relieve one or more of the symptoms associated with carpal tunnel syndrome. Yet another object of the invention is to provide a transdermal drug delivery system that has an improved flux of anesthetic agents and steroids compared to a system of equal size.

It is also an object of the invention to provide a dosage form for transdermal delivery in which the containing pharmaceutical compounds such as anesthetic agents, steroids, and NSAIDs are stable upon storage.

In accomplishing the foregoing and other objectives, there has been provided according to one aspect of the present invention a composition, preferably a dermal composition, resulting from an admixture that includes: a combination of therapeutically effective amount of synthetic local anesthetic and/or synthetic steroids or steroid derivatives, and/or non-steroidal anti-inflammatory agents; and a pharmaceutically acceptable carrier. In a preferred embodiment, the carrier is a polymer that includes a pressure-sensitive adhesive. A preferred polymeric adhesive is a member selected among the group consisting of acrylic polymers and copolymers. According to another preferred embodiment, the carrier comprises a pressure-sensitive adhesive that includes two or more polymers, and wherein the permeation of the drugs can be adjusted by changing the type and/or proportions of the two or more polymers. The active ingredients and carriers are formulated into compositions. In such compositions, the compositions are formulated “by weight”. As such, active ingredients and inactive ingredients are mixed into compositions that contain a certain total weight of ingredients (active and inactive). Each component of the formulation/composition contributes a given percentage, by weight, to the total composition. The Examples provide further exemplification in this regard.

According to another aspect of the invention, there has been provided a method of making a composition described above that includes forming a mixture of the drugs (preferably anesthetic and/or synthetic steroids or steroid derivatives, and/or non-steroidal anti-inflammatory agents) and a carrier, and further includes: forming the blend into a polymer matrix; and drying the polymer matrix to remove volatile solvents to form the composition.

According to another aspect of the invention, there has been provided a method of treating a human suffering from carpal tunnel syndrome with a therapeutically effective amount of pharmaceutically active agents (preferably anesthetic and/or synthetic steroids or steroid derivatives, and/or non-steroidal anti-inflammatory agents), that includes the steps of: applying to the skin of a human being, the composition described above; and maintaining the composition in contact with the skin for a predetermined length of time sufficient to administer the therapeutic amount of the pharmaceutically active agents. In a preferred embodiment, the site on which the composition is applied is the volar aspect of the wrist proximal, distal, or directly over the carpal tunnel that contains the target median nerve. In another preferred embodiment, the composition or formulation may be covered with an occlusive or non-occlusive dressing, which protects the composition from mechanical removal and may enhance the transport of the anesthetic and/or synthetic steroids or steroid derivatives, and/or non-steroidal anti-inflammatory agents into the dermis.

Further objects, features and advantages of the present invention will be more readily apparent from the following detailed description of the invention.

### BRIEF DESCRIPTION OF THE DRAWINGS

FIG. 1 shows the in vitro cumulative skin permeation profiles of lidocaine from patches of example embodiments of the present invention.

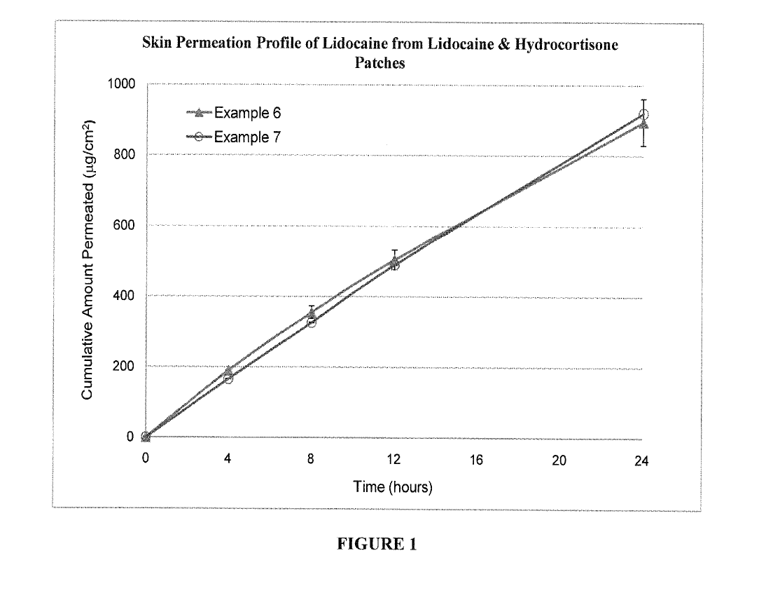

FIG. 2 shows the in vitro cumulative skin permeation profiles of hydrocortisone from patches of example embodiments of the present invention.

### DETAILED DESCRIPTION OF THE DISCLOSURE

Before the present anesthetic and/or synthetic steroids or steroid derivatives, and/or non-steroidal anti-inflammatory agents containing patch device and using the said patch device to treat one or more of the symptoms of a host suffering carpal tunnel syndrome are disclosed and described, it is to be understood that this invention is not limited to the particular process steps and materials disclosed herein as such process steps and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a”, “an”, and “the” include plural referents unless the context clearly dictates otherwise. Thus, for example, reference to an adhesive layer containing “a steroid drug” includes a mixture of two or more steroid drugs, and reference to “an adhesive” includes reference to one or more of such adhesives.

### I. DEFINITIONS AND BACKGROUND INFORMATION

In describing and claiming the present invention, the following terminology will be used in accordance with the definitions set out below.

As used herein, “transdermal” delivery means delivery of a drug by passage into and through the skin or mucosal tissue. Hence the terms “transdermal” and “transmucosal” are used interchangeably unless specifically stated otherwise. Likewise the terms “skin”, “derma”, “epidermis”, “mucosa”, and the like will also be used interchangeably unless specifically stated otherwise.

As used herein, “anesthetics” or “local anesthetic agents” means local anesthetic agents include, and are not limited to, lidocaine, bupivacaine, mepivacaine, dibucaine, prilocaine, etidocaine, ropivacaine, procaine, tetracaine, etc., and mixtures thereof.

As used herein, “steroid drug” or “synthetic steroids” or “synthetic glucocorticoids” means glucocorticoids and include, but are not limited to, hydroxycortisone, cortisone, desoxycorticosterone, fludrocortisone, betamethasone, beclometasone, dexamethasone, prednisolone, prednisone, methylprednisolone, paramethasone, triamcinolone, flumethasone, fluocinolone, fluocinonide, fluprednisolone, halcinonide, flurandrenolide, meprednisone, medrysone, clobetasol, and esters and mixtures thereof.

As used herein, “non-steroidal anti-inflammatory agents” or “NSAIDs” means pharmaceutical agents include, but are not limited to, ketoprofen, ibuprofen, naproxen, indomethacin, sulindac, mefenamic acid, diclofenac, piroxicam, celecoxib, or rofecoxib, acetaminophen, acetylsalicylic acid, and mixtures thereof.

As used herein, “carrier” means a formulated component of a transdermal patch device including, but not limited to, a biocompatible polymeric adhesive, controlled-viscosity composition, penetration enhancer, excipients, diluents, emollient, plasticizer, anti-irritant, opacifier, and the like and mixtures thereof.

As used herein, “matrix”, “matrix system”, or “matrix patch” means a drug intimately admixed, i.e. dissolved or suspended, in a biocompatible polymeric phase, preferably a pressure sensitive adhesive, that can also contain other ingredients or in which an enhancer is also dissolved or suspended. This definition is meant to include embodiments wherein such polymeric phase is laminated to a pressure sensitive adhesive or used with an overlay adhesive. Matrix patches are known in the art of transdermal drug delivery to routinely comprise an impermeable film backing laminated onto the distal surface of the polymeric phase and, before transdermal application, a release liner on the proximal surface of the polymeric phase. A matrix patch according to the present invention should be considered to comprise such backing layer and release liner or their functional equivalents.

The distal backing layer defines the side of the patch that faces the environment, i.e. furthest away from the skin. The functions of the backing layer are to protect the patch and to provide an impenetrable layer or occlusive dressing that prevents loss of the drug to the environment. Thus, the material chosen should be substantially impermeable to the drug. Advantageously, the backing material can be opaque to protect the drug from degeneration from exposure to light. Further, the backing layer should be capable of binding to and supporting the other layers of the patch, yet should be pliable to accommodate the movement of a person using the patch. The layer is preferably of a material that permits the device to mimic the contours of the skin and be worn comfortably on areas of skin, such as at joints or other points of flexion or extension, that are normally subjected to mechanical strain with little or no likelihood of the device disengaging from the skin due to differences in the flexibility or resiliency of the skin and the device. This criterion is particularly critical for the objects of the present invention to treat carpal tunnel syndrome. Elastomeric materials generally present these desired properties. Elastomeric materials that are preferred for use in the practice of the present invention, with or without modification, are those selected from the group consisting of films containing polyester type materials such as Scotchpak™ or Hytrel®, films containing polyether block amide copolymer (e.g. “Pebax®” copolymers), films containing polyurethanes (e.g. “Pellethane°” or “Estane®” polymers), films containing rubber-based polyisobutylene, styrene, styrene-butadiene and styrene-isoprene copolymers, and other such materials used in the art of transdermal drug delivery.

The polymer used in forming the polymer/drug composite should be drug compatible and permit a useful drug flux. The material comprising the polymer layer is preferably a pressure-sensitive skin contact adhesive comprised of a pharmaceutically acceptable material that satisfy the general criteria for adhesives used for transdermal patches including biocompatibility, ease of application, and ease of removal. Suitable adhesives for use include natural and synthetic rubbers including polyisobutylenes, neoprenes, polybutadienes, and polyisoprenes. Cross-linked and uncross-linked acrylic polymers and copolymers are preferred polymeric adhesives for use in the practice of the present invention. Commercially available acrylic polymers and copolymers include GELVA® 737 and GELVA® 788 distributed by Cytec Industries, Inc., Duro-Tak® distributed by National Starch and Chemical Company, Morstik™ 207A and Morstik™ 607 distributed by Dow Chemical Company. These acrylate copolymer materials can be used separately or in mixtures. All of these materials are solvent based but form films following casting and removal of the solvent. These copolymers have the property of being pressure sensitive adhesives when dried and/or cured. Thus the matrices formed from these materials can adhere directly to the patient's skin without the need for additional separate adhesives.

The proximal release liner or peelable film covers the skin-facing side of the device until the device is used. Therefore, the proximal release liner should possess properties similar to those of the backing layer. Just prior to use of the device, the proximal release liner is removed to expose the drug-containing polymer layer for contact and adhesion to the skin. Thus, the proximal release liner is adapted to be removed from the device.

### II. DOSAGE FORMS AND TREATMENT METHODS

The present invention provides for compositions, formulations, methods and systems for relieving one or more of the symptoms of a human suffering from carpal tunnel syndrome by topical delivery of formulations comprising the combination of synthetic local anesthetics and/or synthetic glucocorticoids and/or non-steroidal anti-inflammatory agents applied to the volar aspect of wrist proximal, distal, or directly over the carpal tunnel which contains the target median nerve for a predetermined period of time.

The methods employ using a vehicle which allows for transport of the synthetic local anesthetics and/or synthetic glucocorticoids and/or non-steroidal anti-inflammatory agents, and the mixture thereof across the skin of a patient and achieve an effective concentration of said therapeutic agents to relieve one or more symptoms from carpal tunnel syndrome. The said vehicle includes but is not limited to patches, ointments, creams, gels, solutions, and lotions.

The formulations comprise or consist of synthetic local anesthetics with a range of 0.5% to about 20% by weight and/or synthetic glucocorticoids with a range of 0.1% to about 10% by weight and/or non-steroidal anti-inflammatory agents with a range of 0.5% to about 20% by weight. About 30% to about 90% of the formulation is composed of inactive ingredients (one or more carrier). The said formulation, when applied to in the aforementioned dosage forms, can relieve one or more of the symptoms associated with carpal tunnel syndrome and/or tendonitis. According to embodiments of the present invention, the said topical formulation can mimic the current injection therapy, but do so in a noninvasive and self-administrable manner and thus avoid anxiety, pain, complication and cost associated with injection. Furthermore, another advantage of the present invention over the current practice in art is that a topical formulation can provide more continuous pain and inflammation relief because the formulations and dosage forms provided in the embodiments of the invention can be self-administered more frequently than once every three months, and diminish the potential risk of the tendon damage or rupture with repeated glucocorticoid injections. Still another advantage of the formulation is that the therapeutic effects from the active pharmaceutical agents can be directed to the localized site to avoid systemic absorption and avoid the systemic side effects of glucocorticoids such as hypertension and hyperglycemia.

Composed of acrylate copolymer and the drugs in a matrix patch manner, the drug loaded polyacrylate patch is optimal and preferable in many ways comparing to the current practice or available products:
(i) Since the dosage forms/devices of the invention are externally applied to the skin at or near the carpal tunnel site, the first-pass effect in liver (i.e. decomposition of the drugs occurred when administered orally) can be avoided, resulting in better drug utilization and less systemic side effects comparing to current practice of oral NSAIDs or oral synthetic glucocorticoids.
(ii) Since the dosage forms/devices of the invention are externally applied to the skin at the carpal tunnel site, the therapeutic agents are continuously released for a long period of time, and hence, it is expected that the activity of the drugs is stably exhibited. On the contrary, the current practice of injections of steroids and/or lidocaine to the carpal tunnel space has significant limitations such as: no more than once every 3 months due to risk of tendon rupture and thus there could be significant periods where the patient is not sufficiently medicated; increased pain at the injection site after the anesthetic wears off; infection, bleeding at the injection site; direct needle injury to the median nerve and/or tendon(s); or injection of the drug mixture incorrectly outside of the carpal tunnel space.
(iii) Since the dosage forms/devices of the invention employ formulation of acrylic copolymer matrix systems, utilization rate of lidocaine is significantly higher than the current available lidocaine product such as Lidoderm®. Therefore, it allows for a much smaller patch containing local anesthetics like lidocaine to be effective when it is applied to the wrist area to treat carpal tunnel syndrome, which is much more convenient and comfortable than the larger sized (140 cm²) Lidoderm®. The transdermal patches described in the present invention are also thin and flexible, which become a critical attribute as the wrist is a joint subject to repeated flexion and extension.
(iv) In some embodiments of the present invention, it also shows that lidocaine, when loaded together with glucocorticoids in the acrylic copolymer matrix patches, facilitates the glucocorticoids permeating through skins, which otherwise showed too low permeation to be effective. Glucocorticoids are important for treating carpal tunnel syndrome as it would mitigate the symptoms of carpal tunnel syndrome mediated by inflammation.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

### EXAMPLES

### Example 1

### Preparation of the Transdermal Patches and Stability Samples

A transdermal delivery composition was prepared with the following ingredients:

| Substance | % (by weight) |
|---|---|
| Lidocaine | 5.0 |
| Prednisone | 2.5 |
| Propylene Glycol | 10.0 |
| Gelva ® 737 Adhesive Solution (32.3% polyacrylate) | 82.5 |
| Total | 100.0 |

Preparation of the transdermal patch:
1. Weigh appropriate amounts of active pharmaceutical ingredients (e.g., lidocaine and prednisone), inactive ingredients (e.g., propylene glycol), and adhesive solutions (e.g., Gelva ® 737) accurately in a vessel.
2. Dissolve or suspend the ingredients in the adhesive solution and mix the solution until homogeneous.
3. Place a sheet of release liner onto a patch coater (e.g., Warner Mathis coater)
4. Pour the solution on the release liner and coat a thin film on the release liner.
5. Dry the solution in an oven at preset temperature for a predetermined time to evaporate the solvents.
6. After drying, laminate the dried film with a sheet of backing layer.
7. Cut the laminate with a die cutter into desired sizes.
8. Insert a cut patch into a sealable aluminum pouch.
9. Seal the aluminum pouch with heat and proceed to predetermined stability study.

### Example 2

### In Vitro Skin Permeation Studies

The lidocaine/prednisone patch as described in Example 1 is evaluated to determine the skin permeation of lidocaine. Lidoderm® is included in the study for comparison. Lidoderm® patch was cut into size of 1.5 cm×1.5 cm for the convenience of the skin permeation study. The drug loading of Lidoderm® patch is 700 mg/140 cm².

The in-vitro permeation of lidocaine patch through human cadaver skin was studied using VC (Valia-Chien) skin diffusion cells. The active permeation area for the study was 0.64 cm². Human cadaver skin was cut to desired size and placed on a flat surface of one VC skin diffusion cell with the stratum corneum side facing outward. The release liner was separated from the polyacrylate drug matrix. The drug matrix was placed onto the stratum corneum. The same was repeated for another set of VC skin diffusion cell. The two sets were then clamped together. 10% polyethylene glycol in distill water solution of 3.5 mL was added to the receptor site of the diffusion cell to initiate the skin permeation study. Temperature of the medium was maintained at 37° C. by circulating water from a water bath.

At a predetermined time interval, i.e., 4 hours, 8 hours, 12 hours, and 24 hours, 0.5 mL each of receptor sample was withdrawn. Lidocaine concentration in the samples was assayed by a high-performance liquid chromatography (HPLC) instrument. The cumulative amount of lidocaine in the receptor compartment as a result of skin permeation was calculated and reported. The following table shows the amount of lidocaine released over the time.

| | Cumulative Amount of | Percent |
|---|---|---|
| | Lidocaine Released | Lidocaine Released |
| Lidocaine Patch | (mg/140 cm²) in 12 hours* | in 12 hours* |
| Lidoderm Patch | 32.96 ± 3.87 (n = 3) | 4.7% (n = 3) |
| (700 mg/140 cm²) | | |
| Example 1 | 27.56 ± 3.01 (n = 3) | 53% (n = 3) |
| (52 mg/140 cm²) | | |

*Cumulative amount of lidocaine permeated is reported as mg per 140 cm², which is the original size of Lidoderm ® patch.

### Example 3-11

Transdermal lidocaine/glucocorticoids delivery compositions were prepared according to the manufacturing procedures as described in Example 1 with the following ingredients:

| | Formulation ID | | |
|---|---|---|---|
| Substance | Example 3 | Example 4 | Example 5 |
| Lidocaine | 10.0% | 10.0% | — |
| Hydrocortisone (micronized) | 1.0% | — | 1.0% |
| Starch 1500 | 40.0% | 40.0% | 40.0% |
| Polyacrylate (Gelva ® 737/ | 49.0% | 50.0% | 59.0% |
| Duro-Tak ® 87- | | | |
| 2852 = 85/15) | | | |
| Total | 100.0% | 100.0% | 100.0% |
| | Formulation ID | | |
| Substance | Example 6 | Example 7 | Example 8 |
| Lidocaine | 10.0% | 10.0% | — |
| Hydrocortisone (micronized) | 1.0% | — | 1.0% |
| Starch 1500 | 40.0% | 40.0% | 40.0% |
| Polyacrylate (Gelva ® 737) | 49.0% | 50.0% | 59.0% |
| Total | 100.0% | 100.0% | 100.0% |
| | Formulation ID | | |
| Substance | Example 9 | Example 10 | Example 11 |
| Lidocaine | 10.0% | 10.0% | 10.0% |
| Hydrocortisone (micronized) | 1.0% | 1.0% | 1.0% |
| Starch 1500 | 40.0% | 40.0% | 40.0% |
| Polyacrylate (Gelva ® 737/ | 49.0% | — | — |
| Duro-Tak ® 87- | | | |
| 2074 = 2/8) | | | |
| Polyacrylate (Gelva ® 788/ | — | 49.0% | — |
| Duro-Tak ® 87- | | | |
| 2074 = 2/8) | | | |
| Polyacrylate (Gelva ® 788) | — | — | 49.0% |
| Total | 100.0% | 100.0% | 100.0% |

### Example 12-15

Transdermal lidocaine/glucocorticoids delivery compositions were prepared according to the manufacturing procedures as described in Example 1 with the following ingredients:

| | Formulation ID | | | |
|---|---|---|---|---|
| Substance | Example 12 | Example 13 | Example 14 | Example 15 |
| Lidocaine | 5.0% | 5.0% | 5.0% | 5.0% |
| Hydrocortisone (micronized) | 1.0% | 1.0% | 1.0% | 1.0% |
| Butylated Hydroxytoluene | 1.0% | 1.0% | 1.0% | 1.0% |
| Lauroglycol | — | 1.0% | — | 1.0% |
| Starch 1500 | 43.0% | 42.0% | 43% | 42.0% |
| Polyacrylate (Gelva ® 788) | 50.0% | 50.0% | — | — |
| Polyacrylate (Duro-Tak ® 87- | — | — | 50.0% | 50.0% |
| 2074) | | | | |
| Total | 100.0% | 100.0% | 100.0% | 100.0% |

In vitro skin permeation studies were performed according to the procedures described in Example 2. The results of the cumulative amount of lidocaine and hydrocortisone through skins at 24 hours for Example 3 to 15 are summarized in the following table:

Cumulative Amount Permeated Through Skin

| | Skin | Skin Permeation of |
|---|---|---|
| | Permeation of Lidocaine | Hydrocortisone |
| Formulation ID | (μg/cm²/24 hr) | (μg/cm²/24 hr) |
| Example 3 | 690.9 ± 18.7 | 10.5 ± 2.4 |
| Example 4 | 878.3 ± 45.4 | — |
| Example 5 | — | 9.2 ± 2.3 |
| Example 6 | 896.3 ± 65.6 | 3.8 ± 0.3 |
| Example 7 | 920.6 ± 0.3 | — |
| Example 8 | — | 2.3 ± 0.2 |
| Example 9 | 326.4 ± 71.5 | 22.8 ± 2.3 |
| Example 10 | 301.6 ± 17.8 | 9.4 ± 0.2 |
| Example 11 | 469.7 ± 12.9 | 15.2 ± 1.1 |
| Example 12 | 593.5 ± 97.2 | 11.1 ± 4.4 |
| Example 13 | 796.0 ± 92.2 | 17.5 ± 24.2 |
| Example 14 | 297.5 ± 8.5 | 11.1 ± 1.4 |
| Example 15 | 386.7 ± 104.8 | 13.1 ± 12.6 |

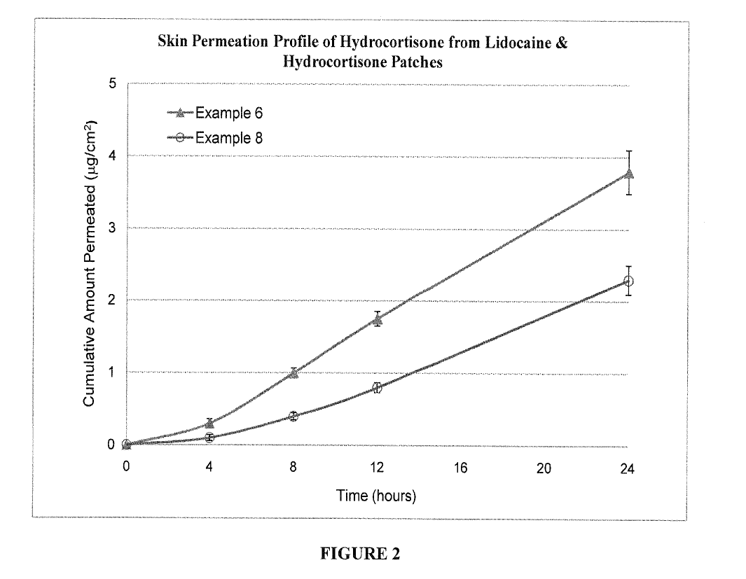

From the in vitro skin permeation study summary, the results indicate with 10% lidocaine added in the formulation, hydrocortisone showed a higher skin flux, which verified the findings of the present invention that lidocaine can facilitate steroid drugs skin permeation. It is also shown the higher flux on FIG. 2. These results are not known or studied in prior art.

The results also indicate that the skin permeation of lidocaine and hydrocortisone is different when these drugs are formulated in different adhesives. Therefore, the suitable and effective composition or formulation containing local anesthetics (e.g. lidocaine) and/or steroids (e.g. hydrocortisone) applicable for treating carpal tunnel syndrome is not obvious to those skilled in art of the pertained area.

The results further showed that permeation enhancers can also facilitate the skin permeation of both lidocaine and hydrocortisone.

### Example 16-19

Transdermal lidocaine/glucocorticoids delivery compositions were prepared according to the manufacturing procedures as described in Example 1 with the following ingredients:

| | Formulation ID | | | |
|---|---|---|---|---|
| Substance | Example 16 | Example 17 | Example 18 | Example 19 |
| Lidocaine | 10.0% | 10.0% | 10.0% | 5.0% |
| Hydrocortisone Acetate | 1.0% | 1.0% | 1.0% | 1.0% |
| (micronized) | | | | |
| Starch 1500 | 40.0% | 40.0% | 40% | 42.0% |
| Polyacrylate (Gelva ® 737) | 49.0% | — | — | — |
| Polyacrylate (Gelva ® 737/Duro- | — | 49.0% | — | — |
| Tak ® 87-2074 = 85/15) | | | | |
| Polyacrylate (Gelva ® 737/Duro- | — | — | 49.0% | — |
| Tak ® 87-2074 = 2/8) | | | | |
| Polyacrylate (Gelva ® 737/Duro- | — | — | — | 49.0 |
| Tak ® 87-2074 = 1/9) | | | | |
| Total | 100.0% | 100.0% | 100.0% | 100.0% |

In vitro skin permeation studies were performed according to the procedures described in Example 2. The results of the cumulative amount of lidocaine and hydrocortisone acetate through skin at 24 hours for Example 16 to 19 are summarized in the following table:

| | Cumulative Amount Permeated Through Skin | |
|---|---|---|
| | | Skin Permeation of |
| | Skin Permeation of Lidocaine | Hydrocortisone Acetate |
| Formulation ID | (μg/cm²/24 hr) | (μg/cm²/24 hr) |
| Example 16 | 1,585.1 ± 108.2 | 1.5 ± 0.6 |
| Example 17 | 1,125.7 ± 17.8 | 3.6 ± 0.9 |
| Example 18 | 626.2 ± 54.3 | 0.77 ± 0.02 |
| Example 19 | 508.4 ± 55.7 | 0.76 ± 0.09 |

### Example 20-22

Transdermal lidocaine/glucocorticoids delivery compositions were prepared according to the manufacturing procedures as described in Example 1 with the following ingredients:

| | Formulation ID | | |
|---|---|---|---|
| Substance | Example 20 | Example 21 | Example 22 |
| Lidocaine | 5.0% | 5.0% | 10.0% |
| Triamcinolone Acetonide | 1.0% | — | — |
| Clobetasol Propionate | — | 1.0% | — |
| Betamethasone Dipropionate | — | — | 1.0% |
| Butylated Hydroxytoluene | 1.0% | 1.0% | 1.0% |
| Lauroglycol | 1.0% | 1.0% | 1.0% |
| Starch 1500 | 42.0% | 42.0% | 42.0% |
| Polyacrylate (Duro-Tak ® 87- | 50.0% | 50.0% | 50.0% |
| 2074) | | | |
| Total | 100.0% | 100.0% | 100.0% |

In vitro skin permeation studies were performed according to the procedures described in Example 2. The results of the cumulative amount at 24 hours for Example 20 to 22 are summarized in the following table:

| Cumulative | | | |
|---|---|---|---|
| Amount Permeated Through | Formulation ID | | |
| Skin (μg/cm²/24 hr) | Example 20 | Example 21 | Example 22 |
| Lidocaine | 431.0 ± 0.7 | 247.9 ± 28.7 | 279.5 ± 48.8 |
| Triamcinolone Acetonide | 12.7 ± 0.7 | — | — |
| Clobetasol Propionate | — | 0.41 ± 0.08 | — |
| Betamethasone Dipropionate | — | — | 0.36 ± 0.25 |

### Example 23

Samples sealed in aluminum pouches from some of the aforementioned examples were stored in stability chambers having storage conditions of 25° C./60% relative humidity (RH), 30° C./65% RH, and 40° C./75% RH. At predetermined time intervals, samples were removed from the stability chambers and extracted with methanol. Methanolic extract was assayed for drug contents by HPLC. Drug contents versus storage times were summarized in the following tables to determine the stability of drugs in the patch products.

Content percentage of Lidocaine (LID) and Hydrocortisone (HCT) in stability samples of Example 9:

| Storage | Initial | | 1 Month | |
|---|---|---|---|---|
| Conditions | LID | HCT | LID | HCT |
| 30° C./65% RH | 106.2 ± 7.8 | 107.9 ± 8.2 | 103.5 ± 5.5 | 76.2 ± 3.6 |
| 40° C./75% RH | | | 94.4 ± 1.8 | 35.6 ± 0.7 |

Content percentage of Lidocaine (LID) and Hydrocortisone (HCT) in stability samples of Example 10:

| Storage | Initial | | 1 Month | |
|---|---|---|---|---|
| Conditions | LID | HCT | LID | HCT |
| 30° C./65% RH | 95.3 ± 3.3 | 98.4 ± 3.6 | 94.4 ± 4.8 | 79.7 ± 4.4 |
| 40° C./75% RH | | | 93.9 ± 2.2 | 46.5 ± 0.7 |

Content percentage of Lidocaine (LID) and Hydrocortisone Acetate (HCA) in stability samples of Example 17:

| Storage | Initial | | | |
|---|---|---|---|---|
| Conditions | LID | HCA | LID | HCA |
| | | | 1 Month | |
| 30° C./65% | 101.6 ± 3.5 | 108.4 ± 3.7 | 102.0 ± 6.9 | 109.4 ± 8.5 |
| RH | | | | |
| 40° C./75% | | | 97.8 ± 4.7 | 106.3 ± 5.6 |
| RH | | | | |
| | | | 2 Month | |
| 30° C./65% | 101.6 ± 3.5 | 108.4 ± 3.7 | 101.5 ± 4.3 | 110.3 ± 4.6 |
| RH | | | | |
| 40° C./75% | | | 95.1 ± 2.0 | 106.1 ± 2.4 |
| RH | | | | |

Content percentage of Lidocaine (LID) and Triamcinolone Acetonide (TAA) in stability samples of Example 20:

| Storage | Initial | | 1 Month | |
|---|---|---|---|---|
| Conditions | LID | TAA | LID | TAA |
| 25° C./60% | 103.4 ± 3.9 | 111.3 ± 3.9 | 103.4 ± 3.9 | 111.3 ± 3.9 |
| RH | | | | |
| 40° C./75% | | | 92.7 ± 4.7 | 98.5 ± 4.7 |
| RH | | | | |

Recovery of the one month and two months stability samples clearly showed that both lidocaine and hydrocortisone acetate are stable in the provided formulation of the present invention. However, recovery of hydrocortisone in one month stability samples from several formulations all indicated that hydrocortisone is not stable in the formulation. Therefore, the stable topical formulation containing local anesthetics (e.g. lidocaine) and/or steroids (e.g. hydrocortisone or hydrocortisone acetate) needs to be carefully studied and screened. The compositions having acceptable stability are not apparent to those skilled in the art to which the invention pertains. Only the compositions and formulations having acceptable stability can be used for treating carpal tunnel syndrome.

### Example 25

### In Vivo Skin Permeation Study

The transdermal patch as described in Example 1 is evaluated to determine the skin permeation of lidocaine in vivo. Lidoderm® patch is included in the study for comparison purposes.

The transdermal patch prepared according to Example 1 with a size of 70 cm² was worn by a volunteer for 12 hours. Both the used and unused patch was extracted with methanol. The amount of lidocaine was assayed by HPLC in all samples. The results showed that the amount of lidocaine in the unused and used patches were 25.6 mg and 11.5 mg, respectively, representing 14.1 mg of lidocaine being released from the patch. The 14.1 mg represents about 55% of lidocaine being released in 12 hours. In comparison, approximately 5% of lidocaine is absorbed after 12 hours of administration of Lidoderm® patch on the skin.

Both in vivo and in vitro permeation study results indicate significant enhancement of bioavailability for the lidocaine transdermal patch of the present invention. It appears that polyacrylate-based lidocaine patch provides enhanced bioavailability and minimized skin irritation. Lidocaine is a lipophilic drug, and it is more soluble in polyacrylate-based adhesive than aqueous-based hydrogel polymer that is used in Lidoderm® patch. The enhanced bioavailability is likely due to the fact that more soluble drug can be made available to partition onto the skin surface. Polyacrylate-based patch is thinner and more patient compliant than hydrogel-based patch. In addition, preservatives are not required in a polyacrylate-based adhesive patch, while it is necessary in an aqueous-based hydrogel patch.

### Example 26

A transdermal delivery composition was prepared according to the manufacturing procedures as described in Example 1, with the following ingredients:

| Substance | % (by weight) |
|---|---|
| Lidocaine | 5.0 |
| Triamcinolone Acetonide | 1.0 |
| Propylene Glycol | 10.0 |
| Gelva ® 737 Adhesive solution (32.3% polyacrylate) | 82.5 |
| Total | 100.0 |

The said transdermal patch is evaluated to determine the skin permeation of lidocaine and triamcinolone in vivo.

The transdermal patch with composition of Example 26 was cut into size of 3 cm² unit patches. The unit patches were worn by four volunteers for 24 hours at the palmar side of the wrists. Both the used and unused patch was extracted with methanol. The amount of lidocaine and triamcinolone was assayed by HPLC in all samples. The results of the in vivo skin permeation of lidocaine and triamcinolone acetonide are summarized in the following table:

| Amount | | | | | |
|---|---|---|---|---|---|
| permeated | | | | | |
| through | | | | | Average ± |
| skin | Subject 1 | Subject 2 | Subject 3 | Subject 4 | S.E. |
| Lidocaine | 276.4 | 299.0 | 256.4 | 214.8 | 261.0 ± 35.4 |
| (μg/cm²/ | | | | | |
| 24 hrs) | | | | | |
| Triamcin- | 7.5 | 8.7 | 9.1 | 13.9 | 9.8 ± 2.9 |
| olone | | | | | |
| Acetonide | | | | | |
| (μg/cm²/ | | | | | |
| 24 hrs) | | | | | |

The data clearly shows that both in vivo and in vitro permeation study results correlate very well. It appears that the compositions provided in the present invention improved bioavailability of lidocaine and the enhanced flux of lidocaine further facilitate the permeation of steroid drugs. Therefore these compositions can deliver both therapeutic agents through skins when applied to or near the site of carpal tunnel of the target median nerve, which is preferred in order to relieve one or more of the symptoms.

### Example 27

A transdermal delivery composition was prepared according to the manufacturing procedures as described in Example 1, with the following ingredients:

| Substance | % (by weight) |
|---|---|
| Lidocaine | 5.0 |
| Diclofenac | 2.5 |
| Propylene Glycol | 10.0 |
| Gelva ® 737 Adhesive solution (32.3% polyacrylate) | 82.5 |
| Total | 100.0 |

### Example 28

A transdermal delivery composition was prepared according to the manufacturing procedures as described in Example 1, with the following ingredients:

| Substance | % (by weight) |
|---|---|
| Lidocaine | 5.0 |
| Hydrocortisone Acetate | 2.5 |
| Diclofenac | 2.5 |
| Propylene Glycol | 10.0 |
| Gelva ® 737 Adhesive solution (32.3% polyacrylate) | 80.0 |
| Total | 100.0 |

What is claimed is:

1. A method for relieving one or more symptoms of carpal tunnel syndrome, said method comprising administering to a subject a topical delivery system comprising a polyacrylate formulation comprising a combination of synthetic local anesthetics and/or synthetic glucocorticoids and/or non-steroidal anti-inflammatory agents.

2. The method according to claim 1, wherein the delivery system further comprises a co-solvent, solubilizer and/or penetration enhancer.

3. The method according to claim 1, wherein the delivery system is selected from a topical patch, ointment, cream, gel, solution, or lotion.

4. The method according to claim 3, wherein the delivery system is a topical patch comprising a backing layer, an adhesive drug matrix with active and inactive ingredients, and a release liner.

5. The method according to claim 4, wherein the active ingredient comprises a combination of synthetic local anesthetics and/or synthetic glucocorticoids and/or non-steroidal anti-inflammatory agents.

6. The method according to claim 5, wherein the synthetic local anesthetic are selected from lidocaine, bupivacaine, mepivacaine, dibucaine, prilocalne, etidocaine, ropivacaine, procaine, tetracaine, or mixtures thereof.

7. The method according to claim 5, wherein the synthetic glucocorticoid is selected from hydroxycortisone, cortisone, desoxycorticosterone, fludrocortisone, betamethasone, beclometasone, dexamethasone, prednisolone, prednisone, methylprednisolone, paramethasone, triamcinolone, flumethasone, fluocinolone, fluocinonide, fluprednisolone, halcinonide, flurandrenolide, meprednisone, medrysone, clobetasol, or esters and mixtures thereof.

8. The method according to claim 5, wherein the non-steroidal anti-inflammatory agents are selected from ketoprofen, ibuprofen, naproxen, indomethacin, sulindac, mefenamic acid, diclofenac, piroxicam, celecoxib, or rofecoxib, acetaminophen, acetylsalicylic acid, or mixtures thereof.

9. The method according to claim 5, wherein the synthetic local anesthetic comprises about 0.5% to about 20% by weight of the polyacrylate composition.

10. The method according to claim 5, wherein the synthetic glucocorticoids comprises about 0.1% to about 10% by weight of the polyacrylate composition.

11. The method according to claim 5, wherein non-steroidal anti-inflammatory agent comprises about 0.5% to about 20% by weight of the polyacrylate composition.

12. The method according to claim 2, wherein the co-solvents, solubilizers and penetration enhancers are sulfoxides, alcohols, polyols, alkanes, fatty acids, esters, amines and amides, terpenes, surface-active agents, biodegradable penetration enhancers or cyclodextrins.

13. The method according to claim 1, wherein the formulation comprises about 30% to about 90% inactive ingredient by weight.

14. The method according to claim 4, wherein the adhesive is pressure sensitive and is selected from kayara-, rubber-, polyacrylate-, and polydimethyl siloxane (silicone)-based adhesive, hydrophilic adhesive compositions, or “hydrogels” composed of high molecular weight polyvinyl pyrrolidone and oligometric polyethylene oxide.

15. The method according to claim 1, wherein the combination or synthetic local anesthetics and/or synthetic glucocorticoids and/or non-steroidal anti-inflammatory agents is:
a) synthetic local anesthetics and synthetic glucocorticoids;
b) synthetic local anesthetics and non-steroidal anti-inflammatory agents;
c) synthetic glucocorticoids and non-steroidal anti-inflammatory agents; or
d) synthetic local anesthetics and synthetic glucocorticoids and non-steroidal anti-inflammatory agents.

16. A method for relieving one or more symptoms of tendonitis, said method comprising administering to a subject a topical delivery system comprising polyacrylate formulations comprising the combination of synthetic local anesthetics and/or synthetic glucocorticoids and/or non-steroidal anti-inflammatory agents.

17. The method according to claim 16, wherein the delivery system further comprises a co-solvent, solubilizer and/or penetration enhancer.

18. The method according to claim 16, wherein the delivery system is selected from a topical patch, ointment, cream, gel, solution, or lotion.

19. The method according to claim 18, wherein the delivery system is a topical patch comprising a backing layer, an adhesive drug matrix with active and inactive ingredients, and a release liner.

20. The method according to claim 19, wherein the active ingredient comprises a combination of synthetic local anesthetics and/or synthetic glucocorticoids and/or non-steroidal anti-inflammatory agents.

21. The method according to claim 16, wherein the synthetic local anesthetic are selected from lidocaine, bupivacaine, mepivacaine, dibucaine, prilocaine, etidocaine, ropivacaine, procaine, tetracaine, or mixtures thereof.

22. The method according to claim 16, wherein the synthetic glucocorticoid is selected from hydroxycortisone, cortisone, desoxycorticosterone, fludrocortisone, betamethasone, beclometasone, dexamethasone, prednisolone, prednisone, methylprednisolone, paramethasone, triamcinolone, flumethasone, fluocinol one, fluocinonide, fluprednisolone, halcinonide, flurandrenolide, meprednisone, medrysone, clobetasol, or esters and mixtures thereof.

23. The method according to claim 16, wherein the non-steroidal anti-inflammatory agents are selected from ketoprofen, ibuprofen, naproxen, indomethacin, sulindac, mefenamic acid, diclofenac, piroxicam, celecoxib, or rofecoxib, acetaminophen, acetylsalicylic acid, or mixtures thereof.

24. The method according to claim 16, wherein the synthetic local anesthetic comprises about 0.5% to about 20% by weight of the polyacrylate formulation.

25. The method according to claim 16, wherein the synthetic glucocorticoid comprises about 0.1% to about 10% by weight of the polyacrylate formulation.

26. The method according to claim 16, wherein the non-steroidal anti-inflammatory agent comprises about 0.5% to about 20% by weight of the polyacrylate composition.

27. The method according to claim 17, wherein the co-solvents, solubilizers and penetration enhancers are sulfoxides, alcohols, polyols, alkanes, fatty acids, esters, amines and amides, terpenes, surface-active agents, biodegradable penetration enhancers or cyclodextrins.

28. The method according to claim 16, wherein the formulation comprises inactive ingredient at about 30% to about 99% by weight.

29. The method according to claim 19, wherein the adhesive is pressure sensitive and is selected from kayara-, rubber-, polyacrylate-, and polydimethyl siloxane (silicone)-based adhesive, hydrophilic adhesive compositions, or “hydrogels” composed of high molecular weight polyvinyl pyrrolidone and oligometric polyethylene oxide.

30. The method according to claim 16, wherein the combination or synthetic local anesthetics and/or synthetic glucocorticoids and/or non-steroidal anti-inflammatory agents is:
a) synthetic local anesthetics and synthetic glucocorticoids;
b) synthetic local anesthetics and non-steroidal anti-inflammatory agents;
c) synthetic glucocorticoids and non-steroidal anti-inflammatory agents; or
d) synthetic local anesthetics and synthetic glucocorticoids and non-steroidal anti-inflammatory agents.
